(19) United States
(12) Patent Application Publication
(10) Pub. No.: US 2011/0020327 A1
(43) Pub. Date: Jan. 27, 2011

(54) Purification of proteins

(75) Inventors: Wilson Moya, Concord, MA (US); Jad Jaber, Sudbury, MA (US)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 12/592,744

(22) Filed: Dec. 2, 2009

Related U.S. Application Data
(60) Provisional application No. 61/201,880, filed on Dec. 16, 2008.

(51) Int. Cl. A61K 39/395; C07K 1/14; C07K 16/18; C07K 16/30; C07K 16/22; C07K 14/62; C07K 14/61; C12N 9/96; C07K 14/505; A61K 38/43; A61K 38/00

(52) U.S. Cl. 424/133.1; 530/412; 530/387.1; 530/388.1; 530/387.3; 530/389.1; 530/399; 435/188; 530/395; 424/141.1; 424/94.3; 514/1.1

(57) ABSTRACT

The present invention relates to a bimodal polymer such as a soluble polymer capable of irreversibly binding to insoluble particulates and a subset of soluble impurities and also capable of reversibly binding to one or more desired biomolecules in an unclarified biological material containing stream and the methods of using such a material to purify one or more desired biomolecules from such a stream without the need for prior clarification. Such a polymer comprises domains of charged pendant groups such as primary, secondary, tertiary or quaternary amines, (first mode) and is rendered insoluble and precipitates out of solution simply upon complexing with oppositely charged solid particulates and a fraction of the soluble impurities in an amount sufficient to form an aggregate that can no longer be held in solution. The polymer further comprises other domains of pendant groups that are charged or uncharged, hydrophilic or hydrophobic or have a ligand that is selective for the biomolecule of interest depending on the process conditions such as pH, ionic strength, salts, and the like (second mode). When present in one mode, such as the uncharged form, said pendant groups are capable of binding to one or more desired biomolecules within the stream (protein, polypeptide, etc) in an unclarified cell broth. The precipitate can then be removed from the stream, such as by being filtered out from the remainder of the stream and the desired biomolecule is recovered such as by selective elution.

### CROSS-REFERENCED TO RELATED APPLICATIONS

This application claims the benefit of U.S. Provisional Patent Application No. 61/201,880, filed on Dec. 16, 2008 the entire contents of which are incorporated by reference herein.

The present invention relates to the purification of biomolecules. More particularly, it relates to the purification of biomolecules such as proteins, polypeptides, antibodies and the like, by a polymer, such as a solubilized or soluble polymer to capture the desired biomolecules from an unclarified cell culture broth by a precipitation mechanism and then to further purify it.

### BACKGROUND OF THE INVENTION

The general process for the manufacture of biomolecules, such as proteins, antibodies, antibody fragments, peptides, polypeptides and the like, particularly recombinant proteins, typically involves two main steps: (1) the expression of the protein in a host cell, followed by (2) the purification of the biomolecule. The first step involves growing the desired host cell in a bioreactor or fermentor to effect the expression of the protein. Some examples of cell lines used for this purpose include Chinese hamster ovary (CHO) cells, myeloma (NSO) bacterial cells such as e-coli and insect cells.

Once the protein is expressed at the desired levels, the biomolecule is removed from the host cell and harvested. In some systems the biomolecule is expressed from the cell and is in the broth. In others, the biomolecule either in not expressed and remains within the cell or is in fact part of the cell and the cell therefore needs to be lysed and in some cases further processed so that the biomolecule can be recovered. Suspended particulates, such as cells, cell fragments, lipids and other insoluble matter are typically removed from the biomolecule-containing fluid by filtration or centrifugation, resulting in a clarified fluid containing the biomolecule of interest in solution as well as other soluble impurities and smaller particulates.

The second step involves the purification of the harvested biomolecule to remove impurities which are inherent to the process. Examples of impurities include host cell proteins (HCP, proteins other than the desired or targeted protein), nucleic acids, endotoxins, viruses, biomolecule variants and biomolecule aggregates.

This purification typically involves several chromatography steps, which can include affinity, ion exchange hydrophobic interaction, etc on solid matrices such as porous agarose, polymeric or glass.

One example of chromatography process train for the purification of proteins involves protein-A affinity, followed by cation exchange, followed by anion exchange. The protein-A column captures the protein of interest or target protein by an affinity mechanism while the bulk of the impurities pass through the column to be discarded. The protein then is recovered by elution from the column. Since most of the proteins of interest have isoelectric points (PI) in the basic range (8-9) and therefore being positively charged under normal processing conditions (pH below the PI of the protein), they are bound to the cation exchange resin in the second column. Other positively charged impurities are also bound to this resin. The protein of interest is then recovered by elution from this column under conditions (pH, salt concentration) in which the protein elutes while the impurities remain bound to the resin. The anion exchange column is typically operated in a flow through mode, such that any negatively charged impurities are bound to the resin while the positively charged protein of interest is recovered in the flow through stream. This process results in a highly purified and concentrated protein solution.

Other alternative methods for purifying proteins have been investigated in recent years; one such method involves a flocculation technique. In this technique, a soluble polyelectrolyte is added to unclarified cell culture broth to capture the suspended particulates and soluble impurities thereby forming a flocculent. The latter is subsequently removed from the biomolecule-containing solution by filtration or centrifugation, resulting in a clarified fluid containing the biomolecule of interest in solution as well as other soluble impurities and some smaller particulates.

Alternatively, a soluble polyelectrolyte is added to clarified cell culture broth to capture the biomolecules of interest, thereby forming a flocculent, which is allowed to settle and can be subsequently isolated from the rest of the solution. The precipitate is typically washed to remove loosely adhering impurities. Afterwards, a change in the solution conditions (pH or ionic strength) brings about the dissociation of the flocculent and subsequent elution of the target biomolecule.

The main drawback of this flocculation technique is that it requires that the polyelectrolyte be added in the exact amount needed to remove the impurities or capture the biomolecule of interest. If too little flocculent is added, impurities or a fraction of the target protein will remain in solution. On the other hand, if too much flocculent is added, the excess polyelectrolyte needs to be removed from the resulting solution. The exact level of impurities in the broth is extremely difficult to predict due to the relatively large degree of variability in the process (from batch to batch) as well as the vast differences between processes to produce different biomolecules. Removing any excess polyelectrolyte is practically impossible because it is a soluble material and thus it is carried through the process as an undesirable impurity.

In co-pending application U.S. Ser. No. 12/004,314 filed Dec. 20, 2007, a polymer, soluble under certain conditions, such as temperature, light, salt levels and/or pH, is used to bind impurities while in its soluble state and is then precipitated out upon a change in condition (pH or temperature, etc) removing the impurities with it. The biomolecule of interest is then further treated using traditional chromatography or membrane adsorbers and the like.

In co-pending application U.S. Ser. No. 12/004,319 filed Dec. 20, 2007 it was suggested that one would the clarification process and chemistries of the application mentioned above to provide one with a clarified feedstock and then use the different chemistries and processes of U.S. Ser. No. 12/004,319, filed Dec. 20, 2007 to purify the biomolecule of interest.

All of the protein purification technologies discussed above share a common theme, and said theme is to first remove suspended particulates and in a second step separate the biomolecules of interest from soluble impurities which are inherent to the process.

In situ product recovery with derivatized magnetic particles is one example of a protein purification technique where the biomolecules of interest can be purified directly from an un-clarified cell culture broth. In this technique, a polymer shell encapsulating a magnetic bead is functionalized with an affinity ligand that seeks out and binds the target protein. A magnetic field is then applied to collect the bead-protein complexes, leaving behind the soluble impurities and insoluble particulates.

The main drawback of this technique is that it requires appreciable capital investments in design, construction and validation of high-gradient magnetic separators. Also, the technique does not lend itself to disposable applications, which are poised to become the norm for protein purification in the Bioprocess industry.

In a co-pending application filed this day, it has been suggested to use the stimulus changing polymers of U.S. Ser. No. 12/004,314 filed Dec. 20, 2007 and U.S. Ser. No. 12/004,319 filed Dec. 20, 2007 with an unclarified broth or liquid and to bind to impurities while in solution and bind or entrain the desired biomolecule as the polymer precipitates out of solution. The precipitate is then separated from the rest of the material, optionally washed and the desired biomolecule is recovered in a purified form such as by selective elution while leaving the polymer and any impurities behind.

The main drawback to this invention is that a stimulus is still needed in order to precipitate the polymer and capture the biomolecule for further processing and purification.

What has been discovered is that a new bimodal polymer can be used in an unclarified feedstock and can recover the biomolecule of interest in a purified form without necessarily going through a stimulus change in order to precipitate thereby providing another new process for the recovery of biomolecules simply and in fewer steps than the traditional methods.

### SUMMARY OF THE INVENTION

The present invention relates to a bimodal polymer such as a soluble polymer capable of irreversibly binding to insoluble particulates and a subset of soluble impurities and also capable of reversibly binding to one or more desired biomolecules in an unclarified biological material containing stream and the methods of using such a material to purify one or more desired biomolecules from such a stream without the need for prior clarification.

Such a polymer comprises domains of charged pendant groups such as primary, secondary, tertiary or quaternary amines, such as quaternized amines, pyridines, imidazoles and triazines (first mode) and is rendered insoluble and precipitates out of solution simply upon complexing with oppositely charged solid particulates and a fraction of the soluble impurities in an amount sufficient to form an aggregate that can no longer be held in solution. The polymer further comprises other domains of pendant groups that are charged or uncharged, hydrophilic or hydrophobic or have a ligand that is selective for the biomolecule of interest depending on the process conditions such as pH, ionic strength, salts, and the like (second mode). When present in one form, for example the uncharged form, said pendant groups are capable of binding to one or more desired biomolecules within the stream (protein, polypeptide, etc) in an unclarified cell broth. The precipitate can then be removed from the stream, such as by being filtered out from the remainder of the stream and the desired biomolecule is recovered such as by selective elution.

The precipitate that contains polymer, impurities and target biomolecule, can be washed one or more times to ensure that any impurities in the liquid or entrapped in or on the polymer have been removed. The biomolecule of interest can be recovered, such as by selective elution of the target molecule from the precipitate by altering the ionic strength and/or pH conditions of the solution while the impurities, including soluble and insoluble material, remain complexed with the precipitated polymer. The purified target biomolecule is recovered in the elution pool and the precipitated polymer-impurity complex is discarded.

It is an object of the present invention to provide a soluble polymer that comprises a mixture of permanently charged pendant groups and reversibly charged pendant groups and that become insoluble and form a precipitate when complexed with soluble and insoluble impurities and the desired biomolecule.

It is an object of the present invention to provide a bimodal polymer that is capable of being selectively solubilized in a liquid under certain conditions and is capable of being rendered insoluble and to precipitate out of solution while complexing with soluble and insoluble impurities and the desired biomolecules.

It is an object to use one or more polymers or copolymers such as polyvinylamine, polyallylamine, poly(diallyldimethylammonium chloride), poly(methacrylamidopropyltrimethylammonium chloride), poly(N-vinyl caprolactam), poly(N-acryloylpiperidine), poly(N-vinylisobutyramide), poly(N-substituted acrylamide) including [poly(N-isopropylacrylamide), poly(N,N′-diethylacrylamide), and poly(N-acryloyl-N′-alkylpiperazine)], Hydroxyalkylcellulose, copolymers of acrylic acid and methacrylic acid, polymers of 2 or 4-vinylpyridine, polymers containing 2 or 4-vinylpyridine and at least one additional monomer and chitosan; with either a ligand or functional group attached to it to selectively capture and reversibly bind to a desired biomolecule in order to purify the biomolecule from a stream containing the biomolecule along with one or more impurities or other entities.

It is an object to use one or more polymers or copolymers of quaternized N-vinyl amine, N-vinyl pyridine, or N-vinyl imidazole with either a ligand or functional group attached to it to selectively capture and reversibly bind to a desired biomolecule in order to purify the biomolecule from a stream containing the biomolecule along with one or more impurities or other entities.

It is another object of the present invention to provide a copolymer selected from poly(N-alkyl 2 or 4-ethynyl pyridinium salt), poly(N-alkyl ethynyl imidazolium salt), poly(N-alkyl ethynyl triazinium salt), polyquaternized amines, and polyquaternized cyclic amines comprising variable ratio's of N-vinyl pyridine, N-acryloylpiperidine, N-vinylisobutyramide or N-substituted acrylamide

It is a further object of the present invention to provide a polymer selected from poly(N-alkyl 2 or 4-ethynyl pyridinium salt), poly(N-alkyl ethynyl imidazolium salt), poly(N-alkyl ethynyl triazinium salt), polyquaternized amines, and polyquaternized cyclic amines wherein the polymer has a ligand such as mercaptoethylpyridine (MEP), mercaptoethylpyrazine, MEB, 2-aminobenzimidazole (ABI), AMBI, 2-mercapto-benzoic acid (MBA), 4-amino-benzoic acid (ABA), 2-mercapto-benzimidazole (MBI), protein A or G and the like, attached to the polymer that selectively binds to the biomolecule of interest

It is a further object of the present invention to provide a process for purifying a selected biomolecule from a biomolecule containing unclarified stream by either having the stream at a given condition or modifying the stream to a given condition and adding a bimodal polymer soluble in the stream at that given condition, allowing the solubilized bimodal polymer to circulate throughout the stream so that the first mode can bind to one or more particulates such as cellular components and soluble impurities and the second mode can reversibly bind to the desired biomolecule, form a precipitate and become insoluble in the stream, separating the stream from the precipitated polymer and processing the polymer further to recover the desired biomolecule by elution while maintaining the polymer with its captured impurities in its precipitated (solid) form.

It is an additional object of the present invention to provide the process based on a polymer which is soluble based upon a condition selected from temperature, salt, temperature and salt content or pH.

It is another object of the present invention to provide a polymer selected from poly(2 or 4-vinylpyridine), poly(2 or 4-vinylpyridine-co-styrene), poly(2 or 4-vinylpyridine-co-methyl methacrylate), poly(2 or 4-vinylpyridine-co-butyl methacrylate), Poly(2 or 4-vinylpyridine) grafted hydroxyalkylcellulose, poly(2 or 4-vinylpyridine-co-N-isopropylacrylamide), and poly(methacrylic acid-co-methylmethacrylate).

It is a further object of the present invention to provide a polymer selected from poly(2 or 4-vinylpyridine), poly(2 or 4-vinylpyridine-co-styrene), poly(2 or 4-vinylpyridine-co-methyl methacrylate), poly(2 or 4-vinylpyridine-co-butyl methacrylate), poly(2 or 4-vinylpyridine) grafted hydroxyalkylcellulose, poly(2 or 4-vinylpyridine-co-N-isopropylacrylamide), and poly(methacrylic acid-co-methylmethacrylate) and wherein the polymer either has a functional group, such as a carboxylated or pyridine group, or a ligand such as protein A, attached to the polymer that selectively binds to the biomolecule of interest.

It is an additional object of the present invention to provide a static mixer for causing the mixture and solubilized polymer to mix and to allow the polymer to bind to the one or more entities.

It is another object of the present invention to provide that the one or more entities are a biomolecule in the mixture.

It is an additional object of the present invention to provide a process for the purification of a mixture of biological constituents in a single step.

It is another object of the present invention to provide a process for the purification of a mixture of biological constituents selected from proteins, polypeptides, monoclonal antibodies, humanized, chimeral or animal monoclonal antibodies polyclonal antibodies, antibody fragments, multispecific antibodies, immunoadhesins, and C_{H}2/C_{H}3 region-containing proteins.

It is a further object of the present invention to provide a process of having a mixture containing a biomolecule of interest at a set range of conditions that will cause one or more polymers of choice to go into solution, adding the one or more polymers and having one or more polymers go into solution, causing the first mode of the polymer to bind to particulates and other impurities while allowing the second mode to reversibly bind to the biomolecule of interest, causing the one or more polymers with the impurities and biomolecule of interest to precipitate out of solution and then separating the precipitate from the remainder of the mixture, while retaining one or more entities of the mixture to the precipitate for further processing.

It is a further object of the present invention to provide a process for recovering a biomolecule of interest from an unclarified mixture obtained from a fermentor or bioreactor in which it has been made.

It is an additional object of the present invention to provide a filtration step to separate the precipitate from the remainder of the mixture.

It is another object of the present invention to provide a normal flow filtration step to separate the precipitate from the remainder of the mixture.

It is a further object of the present invention to provide a tangential flow filtration step to separate the precipitate from the remainder of the mixture.

It is an additional object of the present invention to provide a centrifugation step to separate the precipitate from the remainder of the mixture.

It is another object of the present invention to provide a decantation step to separate the precipitate from the remainder of the mixture.

It is an additional object of the present invention to provide a further step to recover the one or more biomolecules of the mixture from the precipitated polymer by elution under conditions that keep the polymer in its precipitated form.

It is a further object of the present invention to provide additional processing to the biomolecule of interest.

It is an additional object of the present invention to provide a further step of formulating the biomolecule in a pharmaceutically acceptable carrier and using it for various diagnostic, therapeutic or other uses known for such biomolecules.

It is an object of the present invention to provide a purified biomolecule in one step, directly in or out of the bioreactor.

It is a further object of the present invention to use a UF step to concentrate the biomolecule after it has been purified and recovered with the precipitation technique.

It is an additional object of the present invention to effect the purification and recovery of a biomolecule with additional processing using an enhanced UF (charged UF) process.

### IN THE DRAWINGS

FIG. 1 shows a block diagram of a first process according to the present invention.

### DETAILED DESCRIPTION OF THE INVENTION

The invention is to use one or more liquid phase or solubilized bimodal polymers that has/have a capability even when precipitated, such as affinity or charge or hydrophobicity and the like, to bind to particulates and impurities with its first mode and to selectively and reversibly bind to one or more biomolecules of interest with its second mode. Preferred polymers have electrostatic and hydrophobic ability. The biomolecule of interest is then eluted from the polymer preferably while the polymer is retained in its solid or precipitated form and recovered for further processing.

More specifically, the idea relates to the process of using one or more polymers soluble in a liquid phase to use its first mode to bind to particulates and impurities in the liquid and via its second mode to selectively bind to one or more desired biomolecules in a solution, to form a precipitate and to recover the biomolecule from the precipitate. By way of example, this idea can best be described in the context of protein purification although it can be used to purify any solute from complex mixtures as long as the mechanism of removal applies to the specific solute of interest.

Certain polymers, such as poly(N-vinyl caprolactam), poly(N-acryloylpiperidine), poly(N-vinylisobutyramide), poly(N-substituted acrylamide) including [poly(N-isopropylacrylamide), poly(N,N′-diethylacrylamide), and poly(N-acryloyl-N′-alkylpiperazine)] and hydroxyalkylcellulose are examples of polymers that exhibit solubility changes as a result of changes in temperature. Other polymers, such as copolymers of acrylic acid and methacrylic acid, polymers and copolymers of 2 or 4-vinylpyridine and chitosan exhibit changes in solubility as a result of changes in pH or salt. Certain polymers, such as poly(N-alkyl 2 or 4-ethynyl pyridinium salt), poly(N-alkyl ethynyl imidazolium salt), poly(N-alkyl ethynyl triazinium salt), polyquaternized amines, and polyquaternized cyclic amines are examples of polymers that are soluble in aqueous solutions and can complex to oppositely charged impurities

As some of these polymers may not have an inherent second mode ability to selectively bind or elute the desired molecules of interest they need to be modified with ligands or chemical groups that will complex with the desired molecule and hold it in complex and then release the desired molecule under the appropriate elution conditions. Suitable chemical groups can include but are not limited to carboxyl groups and amine groups, such as pyridine groups formed as part of the polymer or attached to the polymer. Ligands such as chemical mimics of affinity ligands may be used. Such ligands include but are not limited to natural ligands or synthetic ligands such as mercaptoethylpyridine (MEP), mercaptoethylpyrazine, MEB, 2-aminobenzimidazole (ABI), AMBI, 2-mercapto-benzoic acid (MBA), 4-amino-benzoic acid (ABA), 2-mercapto-benzimidazole (MBI) and the like.

Depending upon the polymer used, the process used can vary.

Unlike in the prior inventions, one can use unclarified cell culture fluid containing the biomolecule of interest along with cells, cellular debris, host cell proteins, DNA, viruses and the like in the present invention. Moreover, the process can be conducted on harvested cell culture fluid (unclarified cell culture fluid) such as from a bioreactor or it may, if desired, be conducted in the bioreactor itself.

The fluid may either be preconditioned to a desired pH, temperature or other characteristic that allows the polymer(s) to go into solution and have both modes perform their functions or the fluid can be conditioned upon addition of the polymer(s) or the polymer(s) can be added to a carrier liquid that is properly conditioned to the required parameter for that polymer to be solubilized and active in the fluid. The polymer(s) is allowed to circulate thoroughly with the fluid and bind to the impurities and desired biomolecule forming an aggregate that will precipitate out of solution. The polymer, impurities and desired biomolecule(s) is separated from the rest of the fluid and optionally washed one or more times to remove any trapped or loosely bound contaminants. The desired biomolecule is then recovered from the polymer(s) such as by elution and the like. Preferably, the elution is done under a set of conditions such that the polymer remains in its solid (precipitated) form during the elution of the desired biomolecule although both could be solubilized in new fluid such as water or a buffered solution and the biomolecule be recovered by a means such as affinity, ion exchange, hydrophobic, or some other type of chromatography that has a preference and selectivity for the biomolecule over that of the polymer or impurities. The eluted biomolecule is then recovered and if desired subjected to additional processing steps.

Polymers that have the bimodal characteristics include but are not limited quaternized polyvinylpyridine (QPVP). In particular, QPVP where the quaternization is 50% or less are preferred. Other polymers can include polyvinylamine, polyallylamine, poly(diallyldimethylammonium chloride), poly(methacrylamidopropyltrimethylammonium chloride), quaternized polyvinylimidazole, quaternized polyvinyltriazine, quaternized polyamine and quaternized polycyclicamine. These have a first mode that is hydrophilic and/or hydrophobic. They have a second mode mechanism that can be chemical such as a functional group such as a carboxyl or pyridine group such as a quaternized amine group or it may be a ligand such as Protein A, Protein G, synthetic mimics of Protein A such as MEP or MAB.

The processes will generally involve having one or more conditions of the liquid of the mixture, at the correct pH, temperature or salt concentration or other condition used to cause the polymer(s) to become soluble and perform their bimodal functions and then adding the polymer(s) either directly or already solubilized in a carrier liquid, such as water or buffered solution, to the mixture. In some instances, the mixture will be at the proper condition to allow the polymer(s) to be simply added to the mixture.

In other instances, the mixture may need to be conditioned or modified to be at the desired condition. This modification or conditioning can be done by modifying the mixture first and then adding the polymer(s), by adding the polymer(s) to a carrier liquid that is conditioned to the desired state and simply adding it to the mixture such that the carrier liquid is sufficient to cause the mixture to thus reach that condition or to do both.

The polymer's first and second modes bind to their selective targets and form an aggregate that causes the polymer(s) to become insoluble and precipitate out of the mixture as a dispersed solid suspension without the need for a stimulus change.

The precipitate is separated such as by centrifugation or filtration or gravity and time with the liquid portion being decanted. The recovered polymer/desired biomolecule(s) precipitate is washed one or more times to remove any residual impurities or contaminants and then the biomolecule(s) is eluted from the polymer under conditions that cause the biomolecule entity to selectively release from the polymer so it can be recovered and subjected to further processing. Preferably, the elution conditions are such that the polymer remains in its solid or precipitated form while retaining the impurities as well. The eluted biomolecule is separated from the polymer by simple filtration that allows the biomolecule through but retains the polymer upstream.

One polymer or a blend of polymers may be used in the present invention and it is meant to cover both embodiments whenever the term polymer, polymer(s) or one or more polymers is used hereafter.

As discussed above, the polymer may be added directly to the mixture either as is or in a conditioned state that enhances the solubility and binding ability of the first and second modes of the polymer as it is added. Alternatively, it can be added to a carrier liquid in which it is soluble and which carrier preferably is also compatible with the mixture. One such carrier liquid is water, water adjusted to a specific pH using acid or base, another is an aqueous based solution such as saline, physiological buffers or blends of water with an organic solvent such as water/alcohol blends. The selection of carrier liquid is dependent on the mixture to which it is added as to what is preferred and tolerated. The polymer is added to the carrier liquid that either has already been conditioned (such as pH adjusted or heated to a desired temperature or heated to a desired temperature with the addition of one or more salts or cooled to the desired temperature with or without one or more salts) or it can be added and then the carrier is conditioned to cause the solubilizing of the polymer in the carrier. The carrier/soluble polymer blend is then added to the mixture.

The mixture may be contained in a mixing vessel such as a tapered bottom metal (preferably stainless steel more preferably 304 or 316L stainless steel) or glass or plastic bag, vat or tank. Alternatively, especially when a cell culture or microbial or yeast culture, it may be the bioreactor or fermentor in which the cells have been grown. It may also be a disposable bioreactor or fermentor or a disposable mixing bag such as a plastic bag as is available from Millipore Corporation of Billerica, Mass. The mixture and polymer are brought into intimate contact through a mixing action that may be done by a magnetic stirred bar, a magnetic driven mixer such as a NovAseptic® mixer available from Millipore Corporation of Billerica, Mass., a Lightning-type mixer, a recirculation pump, or a rocking motion closed mixing bag or bioreactor or fermentor, such as is shown in US 2005/0063259A1 and U.S. Pat. No. 7,377,686 or an airlift type of mixer or reactor in which rising bubbles in the liquid cause a circulatory pattern to be formed.

Alternatively, the mixture and polymer (either by itself or in a carrier) can be in separate containers and mixed in line in a static blender. The blend can either then go to a container or to a centrifuge or a filter where the precipitated polymer and its bound one or more biomolecule entities is separated from the remainder of the mixture and then is further processed.

In another embodiment, the mixture and polymer (either by itself or in a carrier) are blended together in the container holding the mixture and further mixed in line in a static blender. The blend can either then go to a container or to a centrifuge or to a filter where the precipitated polymer and its bound one or more entities is separated from the remainder of the mixture. Then the precipitated polymer is further processed to recover the biomolecule of interest.

Using centrifugation, one can easily and quickly separate the precipitated polymer from the remainder of the liquid mixture. After centrifugation, the supernatant, generally the remainder of the mixture, is drawn off. The precipitated polymer is further processed to recover the biomolecule.

If desired, the supernatant may be subjected to one or more additional polymer precipitation steps to recover even more of the desired biomolecule.

Simple decantation may also be used if desired.

Filtration can be accomplished in a variety of manners. Depending upon the size of the polymer as it is precipitated; one may use one or more filters of varying sizes or asymmetries. The selection of type and size of filter will depend on the volume of precipitate to be captured.

Membrane based filters, preferably microporous membranes can be used in the present invention. Such filters are generally polymeric in nature and can be made from polymers such as but not limited to olefins such as polyethylene including ultrahigh molecular weight polyethylene, polypropylene, EVA copolymers and alpha olefins, metallocene olefinic polymers, PFA, MFA, PTFE, polycarbonates, vinyl copolymers such as PVC, polyamides such as nylon, polyesters, cellulose, cellulose acetate, regenerated cellulose, cellulose composites, polysulfone, polyethersulfone, polyarylsulfone, polyphenylsulfone, polyacrylonitrile, polyvinylidene fluoride (PVDF), and blends thereof. The membrane selected depends upon the application, desired filtration characteristics, particle type and size to be filtered and the flow desired. Preferred membrane based filters include DURAPORE® PVDF membranes available from Millipore Corporation of Billerica Mass., MILLIPORE EXPRESS® and MILLIPORE EXPRESS® PLUS or SH PES membranes available from Millipore Corporation of Billerica Mass. Prefilters, depth filters and the like can also be used in these embodiments such as Polygard® prefilters (Polygard CE prefilters) and depth filters (Polygard CR depth filters) available from Millipore Corporation of Billerica Mass.

Depending on the mixture, polymer and the nature of biomolecule, the filter may be hydrophilic or hydrophobic. Preferred filters are hydrophilic and are low in protein binding.

The filter, be it membrane or otherwise may be symmetric in pore size throughout its depth such as DURAPORE® PVDF membranes available from Millipore Corporation of Billerica Mass., or it may be asymmetric in pore size through its thickness as with MILLIPORE EXPRESS® and MILLIPORE EXPRESS® PLUS or SH PES membranes available from Millipore Corporation of Billerica Mass. It may contain a prefilter layer if desired, either as a separate upstream layer or as an integral upstream portion of the membrane itself.

The filter or prefilter or depth filter may be formed of non-membrane materials such as continuous wound fiber, fibrous mats (Millistak+® pads) and/or non woven materials such as Tyvek® plastic paper.

The pore size of the membrane can vary depending upon the polymer and mixture selected. Generally, it has an average pore size of from about 0.05 micron to 5 microns, preferably from about 0.05 micron to about 1 micron, more preferably from about 0.05 to about 0.65 micron.

Prefilters and depth filters often are not rated by pore size but to the extent that they are they may have a pore size of from about 0.22 micron to about 10 micron.

The filter, membrane or otherwise may run in a deadend or normal flow (NF) format or a tangential flow (TFF) format. The choice is dependent on a number of factors, primarily the users preference or installed filtration equipment as either works with the present invention. A TFF process and equipment is preferred when large amounts of polymer and molecule are to be recovered as TFF is less subject to clogging or fouling than NF methods.

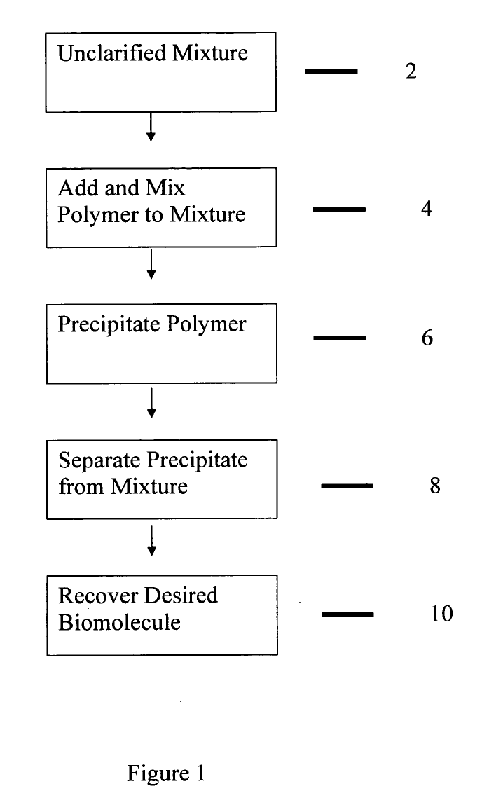

FIG. 1 shows a block diagram of a first process of the present invention. In the first step 2, the unclarified mixture is either conditioned to the correct parameter(s) so as to maintain the capture polymer of choice in solution and allow the two modes to function when added or if the conditions of the mixture are already such that the polymer(s) become soluble and bifunctional in the mixture, no further conditioning may be required. Alternatively, the polymer(s) may be added as a solid to an unconditioned mixture and then the mixture (containing the solid polymer(s)) may be conditioned to the correct parameters to dissolve the polymer(s) in the mixture and allow their bimodal functions to occur. Likewise, the polymer can be added to a carrier liquid and added at the correct conditions to the mixture. The mixture itself may also be preconditioned or it may rely on the carrier to condition it upon its introduction. In the second step 4, the polymer(s) is mixed with the mixture in the stream for desirable amount of time to create suitable distribution to make intimate contact with all the constituents of the mixture. In the third step 6, the polymer(s) form an aggregate and precipitate out of the mixture as a dispersed solid suspension while retaining the bound impurities and the biomolecule. The rest of the mixture and the precipitated polymer(s) are then separated from each other in the fourth step 8. As discussed above the precipitate and remaining mixture may be separated by centrifugation, decantation or filtration.

The precipitate can then optionally be washed one or more times with water, a buffer or an intermediate wash solution as are known in the art to remove any impurities from the precipitate or any non-specifically bound impurities from the precipitate.

The desired biomolecule is then recovered. Preferably it is eluted from the polymer such as by the addition of a buffer at a pH (acidic or basic depending on the molecule and the polymer used) and the salt concentration or temperature of the solution is changed to allow for the recovery of the desired molecule free of the polymer in step 10. Preferably the elution conditions are such that the polymer remains in its solid (precipitated) form and retains the impurities it is bound to although it can if desired or needed be rendered soluble again, the biomolecule decomplexed from the polymer and the polymer can then be reprecipitated such that the first mode works and removes the impurities but the second mode doesn't so that the biomolecule remains with the liquid.

As the biomolecule is recovered with the precipitate, any excess polymer is left with the liquid that is separated from the precipitate thereby reducing any issue with whether any residual polymer remains in the liquid stream.

If desired, one can add additional polymer to the remaining liquid to ensure that as much biomolecule is recovered as possible.

The biomolecule of interest after having been recovered, may undergo one or more known additional process steps such as chromatography steps including but not limited to ion exchange, hydrophobic interaction or affinity chromatography, various filtration steps such as microfiltration, ultrafiltration, high performance tangential flow filtration (HPTFF) with or without charged UF membranes, viral removal/inactivation steps, final filtration steps and the like. Alternatively, the eluted biomolecule of interest may be used as is without the need for further purification steps. Also the biomolecule of interest may undergo further purification without the need for chromatography steps.

In a further embodiment, it eliminates the process steps of cell harvest through affinity chromatography. A biological process under this embodiment would consist of capture of the biomolecule directly from the unclarified mixture via the polymer-based purification step, separation of the biomolecule from the polymer and the remainder of the mixture, two or more steps of viral removal or inactivation such as removal through viral filters or inactivation through treatment with heat, chemicals or light, a compounding step into the correct formulation and a final filtering before filling the compounded biomolecule into its final container for use (vial, syringe, etc).

In any of the embodiments of the present invention the biomolecule such as a protein thus recovered may be formulated in a pharmaceutically acceptable carrier and is used for various diagnostic, therapeutic or other uses known for such molecules.

The mixture that is the starting material of the process will vary depending upon the cell line in which it was grown as well as the conditions under which it is grown and harvested. For example, in most CHO cell processes the cells express the molecule outside of the cell wall into the media. One tries not to rupture the cells during harvest in order to reduce the amount impurities in the mixture. However, some cells during growth and harvesting may rupture due to shear or other handling conditions or die and lyse, spilling their contents into the mixture. In bacteria cell systems, the biomolecule is often kept with the cellular wall or it may actually be part of the cellular wall (Protein A). In these systems, the cell walls need to be disrupted or lysed in order to recover the biomolecule of interest.

The target molecule to be purified can be any biomolecule, preferably a protein, in particular, recombinant protein produced in any host cell, including but not limited to, Chinese hamster ovary (CHO) cells, Per.C6® cell lines available from Crucell of the Netherlands, myeloma cells such as NSO cells, other animal cells such as mouse cells, insect cells, or microbial cells such as E. coli or yeast. Additionally, the mixture may be a fluid derived from an animal modified to produce a transgenic fluid such as milk or blood that contains the biomolecule of interest. Optimal target proteins are antibodies, immunoadhesins and other antibody-like molecules, such as fusion proteins including a C_{H}2/C_{H}3 region. In particular, this product and process can be used for purification of recombinant humanized monoclonal antibodies such as (RhuMAb) from a conditioned harvested cell culture fluid (HCCF) grown in Chinese hamster ovary (CHO) cells expressing RhuMAb.

Antibodies within the scope of the present invention include, but are not limited to: anti-HER2 antibodies including Trastuzumab (HERCEPTIN®) (Carter et al., Proc. Natl. Acad. Sci. USA, 89:4285-4289 (1992), U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108, B1, or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN®. (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58:377-380 (1994)); anti-IgE (Presta et al., J Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al. J. Immunol. 156(4):1646-1653 (1996), and Dhainaut et al. Crit. Care Med. 23(9):1461-1469 (1995)); anti-Tissue Factor (TF) (European Patent No. 0 420 937 B1 granted Nov. 9, 1994); anti-human α₄β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al. Arthritis Rheum 39(1):52-56 (1996)); anti-CD52 antibodies such as CAMPATH-1H (Riechmann et al. Nature 332:323-337 (1988)); anti-Fc receptor antibodies such as the M22 antibody directed against FcγRI as in Graziano et al. J. Immunol. 155(10):4996-5002 (1995); anti-carcinoembryonic antigen (CEA) antibodies such as hMN-14 (Sharkey et al. Cancer Res. 55(23 Suppl): 5935s-5945s (1995); antibodies directed against breast epithelial cells including huBrE-3, hu-Mc 3 and CHL6 (Ceriani et al. Cancer Res. 55(23): 5852s-5856s (1995); and Richman et al. Cancer Res. 55(23 Supp): 5916s-5920s (1995)); antibodies that bind to colon carcinoma cells such as C242 (Litton et al. Eur J. Immunol. 26(1):1-9 (1996)); anti-CD38 antibodies, e.g. AT 13/5 (Ellis et al. J Immunol. 155(2):925-937 (1995)); anti-CD33 antibodies such as Hu M195 (Jurcic et al. Cancer Res 55(23 Suppl):5908s-5910s (1995) and CMA-676 or CDP771; anti-CD22 antibodies such as LL2 or LymphoCide (Juweid et al. Cancer Res 55(23 Suppl):5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1). The preferred target antigens for the antibody herein are: HER2 receptor, VEGF, IgE, CD20, CD11a, and CD40.

Aside from the antibodies specifically identified above, the skilled practitioner could generate antibodies directed against an antigen of interest, e.g., using the techniques described below.

The antibody herein is directed against an antigen of interest. Preferably, the antigen is a biologically important polypeptide and administration of the antibody to a mammal suffering from a disease or disorder can result in a therapeutic benefit in that mammal. However, antibodies directed against non-polypeptide antigens (such as tumor-associated glycolipid antigens; see U.S. Pat. No. 5,091,178) are also contemplated. Where the antigen is a polypeptide, it may be a transmembrane molecule (e.g. receptor) or ligand such as a growth factor. Exemplary antigens include those proteins described in section (3) below. Exemplary molecular targets for antibodies encompassed by the present invention include CD proteins such as CD3, CD4, CD8, CD19, CD20, CD22, CD34, CD40; members of the ErbB receptor family such as the EGF receptor, HER2, HER3 or HERO receptor; cell adhesion molecules such as LFA-1, Mac1, p150,95, VLA-4, ICAM-1, VCAM and αv/β3 integrin including either α or β subunits thereof (e.g. anti-CD11a, anti-CD18 or anti-CD11b antibodies); growth factors such as VEGF; IgE; blood group antigens; flk2/flt3 receptor; obesity (OB) receptor; mpl receptor; CTLA-4; protein C, or any of the other antigens mentioned herein. Antigens to which the antibodies listed above bind are specifically included within the scope herein.

Soluble antigens or fragments thereof, optionally conjugated to other molecules, can be used as immunogens for generating antibodies. For transmembrane molecules, such as receptors, fragments of these (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule.

Other antigens and forms thereof useful for preparing antibodies will be apparent to those in the art.

Polyclonal antibodies can also be purified in the present invention. Polyclonal antibodies are preferably raised in animals by multiple subcutaneous (sc) or intraperitoneal (ip) injections of the relevant antigen and an adjuvant. It may be useful to conjugate the antigen to a protein that is immunogenic in the species to be immunized, e.g., keyhole limpet hemocyanin, serum albumin, bovine thyroglobulin, or soybean trypsin inhibitor using a bifunctional or derivatizing agent, for Example, maleimidobenzoyl sulfosuccinimide ester (conjugation through cysteine residues), N-hydroxysuccinimide (through lysine residues), glutaraldehyde, succinic anhydride, SOCl₂, or R¹N═C NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of antigen or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies are of interest in the present invention and may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For Example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for Example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for Example, Pro-Sep® Protein A media available from Millipore Corporation of Billerica, Mass., hydroxyapatite chromatography, gel electrophoresis, dialysis, or affinity chromatography. Preferably the Protein A chromatography procedure described herein is used.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

The DNA also may be modified, for Example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

In a further embodiment, monoclonal antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional hybridoma techniques for isolation of monoclonal antibodies.

A humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (Sims et al., J. Immunol., 151:2296 (1993)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For Example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and Duchosal et al. Nature 355:258 (1992). Human antibodies can also be derived from phage-display libraries (Hoogenboom et al., J. Mol. Biol., 227:381 (1991); Marks et al., J. Mol. Biol., 222:581-597 (1991); Vaughan et al. Nature Biotech 14:309 (1996)).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For Example, the antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

Multispecific antibodies have binding specificities for at least two different antigens. While such molecules normally will only bind two antigens (i.e. bispecific antibodies, BsAbs), antibodies with additional specificities such as trispecific antibodies are encompassed by this expression when used herein.

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to another approach described in WO96/27011, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For Example, one of the antibodies in the heteroconjugate can be coupled to avidin, the other to biotin. Such antibodies have, for Example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For Example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells over expressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For Example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Alternatively, the antibodies can be “linear antibodies” as described in Zapata et al. Protein Eng. 8(10):1057-1062 (1995). Briefly, these antibodies comprise a pair of tandem Fd segments (V_{H}-C_{H}1-V_{H}-C_{H}1) which form a pair of antigen binding regions. Linear antibodies can be bispecific or monospecific.

Antibodies with more than two valencies are contemplated. For Example, trispecific antibodies can be prepared. Tutt et al. J. Immunol. 147: 60 (1991).

The simplest and most straightforward immunoadhesin design combines the binding domain(s) of the adhesin (e.g. the extracellular domain (ECD) of a receptor) with the hinge and Fc regions of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc domain of immunoglobulin G₁(IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:

AC_{L}-AC_{L}; (a)

AC_{H}-(AC_{H},AC_{L}-AC_{H},AC_{L}-V_{H}C_{H}, or V_{L}C_{L}-AC_{H}); (b)

AC_{L}-AC_{H}-(AC_{L}-AC_{H},AC_{L}-V_{H}C_{H},V_{L}C_{L}-AC_{H}, or V_{L}C_{L}-V_{H}C_{H}) (c)

AC_{L}-V_{H}C_{H}-(AC_{H}, or AC_{L}-V_{H}C_{H}, or V_{L}C_{L}-AC_{H}); (d)

V_{L}C_{L}-AC_{H}-(AC_{L}-V_{H}C_{H}, or V_{L}C_{L}-AC_{H}); and (e)

(A-Y)ₙ-(V_{L}C_{L}-V_{H}C_{H})₂, (f)

wherein each A represents identical or different adhesin amino acid sequences;

V_{L} is an immunoglobulin light chain variable domain;

V_{H} is an immunoglobulin heavy chain variable domain;

C_{L} is an immunoglobulin light chain constant domain;

C_{H} is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom, et al., Mol. Immunol. 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for Example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell 61:1303-1313 (1990); and Stamenkovic et al., Cell 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

In other embodiments, the protein to be purified is one which is fused to, or conjugated with, a C_{H}2/C_{H}3 region. Such fusion proteins may be produced so as to increase the serum half-life of the protein. Examples of biologically important proteins which can be conjugated this way include renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD19, CD20, CD34, and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for Example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

The following Examples are offered by way of illustration and not by way of limitation. The disclosures of all citations in the specification are expressly incorporated herein by reference.

### EXAMPLES

### Example 1

### pH Adjustment of an Unclarified Cell Culture Fluid

Cells derived from a non-expressing Chinese Hamster Ovary (CHO) cell line were grown in a bioreactor (New Brunswick Scientific) to a density of 2×10⁶ cells/ml in 10 L of culture medium and harvested at 64% viability. IgG was spiked to a concentration of 0.8 g/L and the concentrations of host cell proteins (HCP) was 4075 ng/ml. The pH of the fluid was 7.2. The pH of the unclarified cell culture fluid was adjusted to 4.5 using 0.5 ml of 1.0M HCl, prior to the start of the purification process.

### Example 2

### This Example Illustrates the Removal of Residual 4-Vinyl Pyridine Monomer from Poly(4-Vinylpyridine)

Linear poly(4-vinylpyridine), (PVP) MW 200,000 obtained form Scientific Polymer Products, Inc., was spread evenly on a glass dish and placed in a vacuum oven. The atmosphere inside the oven was purged with argon for 5 minutes several times to remove oxygen. The pressure in the oven was reduced to 0.1 in mercury using a mechanical vacuum pump and subsequently the temperature was raised to 120° C. The polymer was subjected to these conditions for a total of 24 hours. During this time, the atmosphere inside the oven was purged with argon for 5 minutes several times. At the end of the heating period, the oven temperature was lowered to room temperature and the oven was purged with argon several times before opening the door. The resulting polymer did not have a noticeable odor, whereas the untreated polymer has a distinct odor of 4-vinyl pyridine monomer. The amount of residual 4-vinyl pyridine monomer present in the treated polymer was not detectable by gel permeation chromatography whereas the untreated polymer had 0.05% (w/w) residual 4-vinyl pyridine monomer

### Example 3

### This Example Illustrates the Synthesis of Quaternized Poly(4-Vinyl Pyridine), QPVP

PVP was purified as described in example 2. Iodoethane, Dimethylformamide and toluene were obtained from Sigma and used as received.

A solution of 5 g (0.047 mol based on monomer repeat unit) of PVP, 2.6 g (0.016 mol) of Iodoethane in 30 ml of DMF was maintained at T=80° C. for 12 hr under a nitrogen atmosphere. After cooling to room temperature, the polymer solution was precipitated in 200 ml toluene. The resulting solid was further washed with 100 ml toluene and then dried in an oven at 70° C. for 24 hr with a yield of 95%. The mole ratio of the reactants was selected such that the product comprises 35 mol % of quaternized pyridine rings.

### Example 4

### This Example Illustrates the Preparation of a QPVP Solution

A 10% (w/w) solution of QPVP was prepared by dissolving 10 g purified QPVP from example 3, in 70 g distilled water and 20 g methanol with continuous agitation for 20 minutes at room temperature. The resulting viscous solution was brown in color.

### Example 5

### This Example Illustrates the Capture of IgG from Un-Clarified Cell Culture Fluid Using QPVP

Sodium perchlorate monohydrate and hydrochloric acid (1.0 M) were obtained from Fisher Scientific. 0.3 g of the QPVP solution from example 4 is added to 10 ml of the un-clarified cell culture fluid from example 1. A precipitate, in the form of a dispersed solid suspension, forms instantly as a result of polymer complexation with insoluble impurities (cells and cell debris), soluble impurities (HCP and DNA) and IgG. The resulting solution is mixed continuously for 10 min after adding 0.75 g sodium perchlorate to enhance the binding of IgG to the polymer. The precipitate is collected by centrifugation (4000 rpm for 1 min) and washed with phosphate buffer (50 mM, 0.2M sodium perchlorate, pH 7.5) to remove loosely-bound impurities. While cells, cell debris and a fraction of soluble impurities remain bound to the precipitate, selective elution of the IgG from the precipitate takes place at pH 4.0 (10 mM sodium acetate, 0.1M) followed by filtration through 0.2μ Durapore® filters. Under these conditions, 95% of the IgG present in the original fluid is bound to the polymer and the IgG recovered upon elution is 85 wt %.

What is claimed:

1) A method for purifying a biomolecule from an unclarified mixture containing impurities comprising:
a. providing the mixture at a set of conditions,
b. adding one or more polymers, the polymer being soluble in said mixture under the set of conditions and having a first mode for binding to one or more of the impurities in the mixture and a second mode that is capable of reversibly and selectively binding to the biomolecule,
c. mixing the one or more solubilized one or more polymers throughout the mixture;
d. precipitating the one or more polymers, one or more impurities and bound biomolecule out of solution from the mixture; and
e. separating the precipitated polymer and bound biomolecule from the mixture.

2) The method of claim 1 wherein in step b, the polymer is added to a solution under a set of conditions within the solution and the solution containing the solubilized polymer is added to the mixture.

3) The method of claim 1 further comprising step (f) wherein the biomolecule is recovered from the polymer.

4) The method of claim 1 wherein the one or more polymers is solubilized at a condition selected from the group consisting of pH, temperature, salt concentration, light, electrical charge and combinations thereof.

5) The method of claim 1 wherein the first mode of the polymer is selected from the group consisting of domains of charged pendant groups and the second mode of the polymer is selected from the group consisting of charged pendant groups, uncharged pendant groups, hydrophilic pendant groups, hydrophobic pendant groups and a ligand that is selective for the biomolecule of interest.

6) The method of claim 1 wherein the first mode of the polymer is selected from the group consisting of primary, secondary, tertiary and quaternary amines and the second mode is selected from the group consisting of charged pendant groups, uncharged pendant groups, hydrophilic pendant groups, hydrophobic pendant groups and a ligand that is selective for the biomolecule of interest.

7) The method of claim 1 wherein the polymer is selected from the group consisting of poly(2 or 4-vinylpyridine), poly(2 or 4-vinylpyridine-co-styrene), poly(2 or 4-vinylpyridine-co-methyl methacrylate), poly(2 or 4-vinylpyridine-co-butyl methacrylate), poly(2 or 4-vinylpyridine) grafted hydroxyalkylcellulose, poly(2 or 4-vinylpyridine-co-N-isopropylacrylamide), and poly(methacrylic acid-co-methylmethacrylate) and wherein the second mode of the polymer is selected from the group consisting of a functional group and a ligand.

8) The method of claim 1 wherein the biomolecule is selected from the group consisting of a protein, a recombinant protein, an antibody, a monoclonal antibody, a recombinant monoclonal antibody, a polyclonal antibody, a humanized antibody and an antibody fragment.

9) The method of claim 1 wherein the biomolecule is an antibody fragment selected from the group consisting of Fab, Fab^{!}, f(ab^{!})₂ and Fv fragments, single-chain antibody molecules diabodies, linear antibodies, bispecific antibodies and multispecific antibodies formed from antibody fragments.

10) The method of claim 1 wherein the biomolecule is an antibody that specifically binds to an antigen selected from the group consisting of CD3, CD4, CD8, CD19, CD20, CD34, CD40, EGF receptor, HER2, HER3, HER4 receptor, LFA-1, Mac1, p150,95, VLA-4, ICAM-1, VCAM, av/b3 integrin, CD11a, CD18, CD11b, VEGF, IgE, flk2/flt3 receptor, obesity (OB) receptor, mpl receptor, CTLA-4 and polypeptide C.

11) The method of claim 1 wherein biomolecule is selected from the group consisting of anti-HER2; anti-CD20; anti-IL-8; anti-VEGF; anti-PSCA; anti-CD11a; anti-IgE; anti-Apo-2 receptor; anti-TNF-α, anti-Tissue Factor(TF); anti-CD3; anti-CD25; anti-CD34; anti-CD40; anti-tac; anti-CD4; anti-CD52; anti-Fc receptor; anti-carcinoembryonic antigen (CEA) antibodies; antibodies directed against breast epithelial cells; antibodies that bind to colon carcinoma cells; anti-CD33; anti-CD22; anti-EpCAM; anti-GpIIb/IIIa; anti-RSV; anti-CMV; anti-HIV; anti-hepatitis; anti-αvβ3; anti-human renal cell carcinoma; anti-human 17-1A; anti-human colorectal tumor; anti-human melanoma; anti-human squamous-cell carcinoma; and anti-human leukocyte antigen (HLA) antibodies.

12) The method of claim 1 wherein biomolecule is an antibody selected from the group consisting of anti-HER2 receptor, anti-VEGF, anti-IgE, anti-CD20, anti-CD11a, and anti-CD40 antibodies.

13) The method of claim 1 wherein the biomolecule is selected from the group consisting of an immunoadhesin and an antibody-like molecule.

14) The method of claim 1 wherein the biomolecule is an antibody-like molecule and the antibody-like molecule is a protein fused to, or conjugated with, a C_{H}2/C_{H}3 region.

15) The method of claim 1 wherein the biomolecule is an antibody-like molecule and the antibody-like molecule is a protein fused to, or conjugated with, a C_{H}2/C_{H}3 region and said protein is selected from the group consisting of rennin; growth hormones; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagons; factor VIIIC; factor IX; tissue factor; von Willebrands factor; Protein C; atrial natriuretic factor; lung surfactant; urokinase; human urine and tissue-type plasminogen activator (t-PA); bombesin; thrombin; hemopoietic growth factor; tumor necrosis factor-alpha and -beta; enkephalinase; RANTES; human macrophage inflammatory protein (MIP-1alpha); serum albumins; Muellerian-inhibiting substance; relaxin A-chain; relaxin B-chain; prorelaxin; mouse gonadotropin-associated peptide; beta-lactamase; DNase; IgE, cytotoxic T-lymphocyte associated antigens (CTLAs); inhibin; activin; vascular endothelial growth factor (VEGF); receptors for hormones or growth factors; Protein A or D; rheumatoid factors; bone-derived neurotrophic factor (BDNF); neurotrophin-3. -4, -5, and -6 (NT-3, NT-4, NT-5, and NT-6), nerve growth factors; platelet-derived growth factor (PDGF); fibroblast growth factors; epidermal growth factor (EGF); transforming growth factors (TGF); insulin like growth factor-I and -II (IGF-I and IGF-II); des(1-3)-IGF-I (brain IGF-I) insulin-like growth factor binding proteins (IGFBPs); CD proteins; erythropoietin; osteoinductive factors; immunotoxins; bone morphogenetic proteins (BMPs); interferons-alpha, -beta, and -gamma; colony stimulating factors (CSFs); interleukins IL-1 to IL-10; superoxide dismutase; T-cell receptors; surface membrane proteins; decay accelerating factor; viral antigens; transport proteins; homing receptors; addressing; regulatory proteins; integrins; tumor associated antigens; and fragments thereof.

16) The method of claim 1 further comprising the step of incorporating the recovered biomolecule into a pharmaceutical formulation.

17) The method of claim 1 wherein the one or more polymers are precipitated by the first mode complexing with oppositely charged solid particulates and a fraction of soluble impurities in an amount sufficient to form an aggregate that can no longer be held in solution.

18) The method of claim 1 wherein the one or more polymers are selected from the group consisting of, polyvinylpyridine, copolymers of vinylpyridine, primary amine containing polymers, secondary amine containing polymers and tertiary amine containing polymers.

19) The method of claim 1 further comprising adding the one or more polymers to a carrier liquid under conditions to cause the one or more polymers to go into solution and adding the carrier liquid and the one or more polymers in solution to the mixture through a static mixer.

20) The method of claim 1 further comprising formulating the recovered biomolecule in a pharmaceutically acceptable carrier.

21) The method of claim 1 further comprising formulating the recovered biomolecule in a pharmaceutically acceptable carrier for a purpose selected from the group consisting of research, diagnostic and therapeutic purposes.

22) The method of claim 1 further comprising the biomolecule is recovered from the polymer and the recovered biomolecule has at least 1 LRV reduction in impurities over the starting mixture.

23) The method of claim 1 wherein the biomolecule is recovered from the polymer by eluting the biomolecule under conditions that cause the polymer to remain in its precipitated form without binding to the biomolecule and separating the precipitated polymer from the biomolecule.

24) The method of claim 1 wherein the polymer is a quaternized polyvinylpyridine wherein the quaternization is 50% or less, the first mode is selected from the group consisting of hydrophilic and hydrophobic moieties and the second mode is selected from the group consisting of a carboxyl group, pyridine group and a ligand.

25) The method of claim 1 wherein the polymer is selected from the group consisting of quaternized polyvinylpyridine, polyvinylamine, polyallylamine, poly(diallyldimethylammonium chloride) and poly(methacrylamidopropyltrimethylammonium chloride), the first mode is the first mode is selected from the group consisting of hydrophilic and hydrophobic moieties and the second mode is selected from the group consisting of a carboxyl group, pyridine group and a ligand.

26) The method of claim 1 wherein the polymer is selected from the group consisting of quaternized polyvinylpyridine, polyvinylamine, polyallylamine, poly(diallyldimethylammonium chloride) and poly(methacrylamidopropyltrimethylammonium chloride), the first mode is the first mode is selected from the group consisting of hydrophilic and hydrophobic moieties and the second mode is selected from the group consisting of a carboxyl group, pyridine group and a ligand selected from the group consisting of Protein A, Protein G, MEP and MAB.
